(19) United States
(12) Patent Application Publication
(10) Pub. No.: US 2004/0042988 A1
(43) Pub. Date: Mar. 4, 2004

(54) Personal care compositions containing highly branched primary alcohol component

(76) Inventors: Kirk Herbert Raney, Houston, TX (US); Carolyn Ann Burnley, Houston, TX (US)

Correspondence Address: Jeffrey Y. Kao, Shell Oil Company, Legal - Intellectual Property, P.O. Box 2463, Houston, TX, 77252-2463, US

(21) Appl. No.: 10/651,006

(22) Filed: Aug. 28, 2003

Related U.S. Application Data
(60) Provisional application No. 60/407,724, filed on Sep. 3, 2002.

(51) Int. Cl. A61K 7/06

(52) U.S. Cl. 424/70.1

(57) ABSTRACT

A personal care composition for topical application to the skin or hair has

(i) a branched primary alcohol component, having from about 8 to about 36 carbon atoms and an average number of branches per molecule of from about 0.7 to about 3.0, the branching comprising methyl and ethyl branches, and the branched primary alcohol component optionally having up to about 3 moles of alkylene oxide per mole of alcohol; and

(ii) a cosmetically-acceptable vehicle.

Advantages of the personal care compositions include, for example, excellent stability, viscosity and rheology characteristics, together with emolliency, application and skin feel benefits.

### FIELD OF THE INVENTION

The present invention relates to a personal care composition for topical application to the skin or hair comprising a highly branched primary alcohol component.

### BACKGROUND OF THE INVENTION

Personal care compositions such as skin moisturizing creams, sunscreens, antiperspirants, shampoos, and the like, commonly contain long chain fatty alcohol compounds. These fatty alcohols are commonly linear, saturated or unsaturated alcohols having from 1 to 50 carbon atoms, preferably from 11 to 36 carbon atoms. Such alcohol compounds are useful for providing skin conditioning benefits such as moisturization, humectancy, emolliency, visual improvement of the skin surface, soothing and softening of the skin, improvement in skin feel and the like. Other benefits afforded by long chain fatty alcohol compounds include viscosity and rheology modification.

Two of the most commonly used long chain fatty alcohols in personal care compositions are stearyl alcohol and cetyl alcohol. Both of these alcohols are linear saturated alcohols having 18 carbon atoms and 16 carbon atoms respectively. These are generally derived from naturally occurring glycerides found in most animal and vegetable fats. Although these alcohols provide useful properties when included in personal care compositions, they are typically supplied and shipped as flakes or some other solid form. This means that they need to be converted to liquids by heating before they can be formulated into personal care compositions.

Other alcohol compounds which are known for use in personal care compositions include the so-called “Guerbet” alcohols, which contain some alkyl branching. Typically, “Guerbet” alcohols are liquid at room temperature. The majority of the branching is at the C2 position on the carbon chain. In addition, the alkyl branches tend to be longer chain branches, such as C₄ and above.

Alcohols bearing the tradename NEODOL, commercially available from The Shell Chemical Company, are synthetic blends of long chain alcohols. For example, NEODOL 45 is a mixture of C₁₄ alcohols and C₁₅ alcohols, the majority of which are linear alcohols. NEODOL 45 is marketed by The Shell Chemical Company primarily as a detergent intermediate, but is also marketed as having emollient properties. NEODOL 45 is semisolid at room temperature, being supplied and shipped in the form of flakes and/or powder, and therefore, like cetyl alcohol and stearyl alcohol, needs to be converted to a liquid before it can be incorporated into a personal care formulation.

U.S. Pat. No. 5,849,960 (Shell Oil Company) discloses a branched primary alcohol composition having 8 to 36 carbon atoms which contains an average number of branches per molecule of at least 0.7, said branching comprising methyl and ethyl branching. These alcohols can subsequently be converted to anionic or nonionic detergents or general surfactants by sulfonation or ethoxylation, respectively, of the alcohol. The detergents produced exhibit useful properties such as high biodegradability and high cold water detergency. The use of these branched alcohols in personal care compositions is not discussed in U.S. Pat. No. 5,849,960.

WO99/18929, WO99/18928 and WO97/39089 (The Procter and Gamble Company) disclose personal cleansing compositions comprising mid-chain branched surfactants. The mid-chain branched surfactants are manufactured from mid-chain branched alcohols. The formulations therein however do not contain mid-chain branched alcohols per se, only the corresponding surfactants. In addition, these documents are concerned with cleansing compositions having relatively high levels of surfactant ingredients.

A need still exists for providing personal care compositions with improved formulation, skin feel, viscosity and application properties. An advantage of the present invention is that the use of a particular branched primary alcohol composition having from 0.7 to 3.0 branches per molecule provides personal care compositions which have excellent emolliency, skin feel, skin softening, application and moisturizing properties together with improved viscosity and rheology characteristics. Another advantage of the particular branched primary alcohols used in the present compositions is that they also exhibit the ability to solubilize a wide variety of skin care ingredients and are highly biodegradable.

### SUMMARY OF THE INVENTION

According to one embodiment of the present invention there is provided a personal care composition for topical application to the skin or hair comprising

(i) a branched primary alcohol component, having from about 8 to about 36 carbon atoms and an average number of branches per molecule of from about 0.7 to about 3.0, said branching comprising methyl and ethyl branches, and said branched primary alcohol component optionally comprising up to about 3 moles of alkylene oxide per mole of alcohol, or said branched primary alcohol component optionally comprising a product made by reacting alkylene oxide with branched primary alcohol in a ratio of up to about 3 moles of alkylene oxide per mole of alcohol; and

(ii) a cosmetically-acceptable vehicle.

According to a further aspect of the present invention there is provided the use of a branched alcohol component for providing emolliency benefits to the skin, wherein the branched primary alcohol component has from about 8 to about 36 carbon atoms and an average number of branches per molecule of from about 0.7 to about 3.0, said branching comprising methyl and ethyl branches.

Another embodiment of the invention relates to a method of caring for skin or hair comprising applying to skin or hair a personal care composition as described herein.

### DETAILED DESCRIPTION OF THE INVENTION

All percentages and ratios used herein are by weight of the total personal care composition, unless otherwise specified.

All publications cited herein are incorporated by reference in their entirety, unless otherwise indicated.

The term “cosmetically-acceptable”, as used herein, means that the compositions, or components thereof, are suitable for use in contact with human skin or hair without undue toxicity, incomparability, instability, allergic response, and the like.

The term “safe and effective amount” as used herein means an amount of a compound, component, or composition sufficient to significantly induce a positive benefit, preferably a positive skin appearance or feel benefit, including independently the benefits disclosed herein, but low enough to avoid serious side effects, i.e. to provide a reasonable benefit to risk ratio, within the scope of sound medical judgement.

### Branched Primary Alcohol Component

A first component of one embodiment of the personal care compositions herein is a branched primary alcohol component having from about 8 to about 36 carbon atoms and an average number of branches per molecule of from about 0.7 to about 3.0, said branching comprising methyl and ethyl branching. In addition, the branched primary alcohol component may optionally comprise up to about 3 moles of alkylene oxide per mole of alcohol.

The personal care compositions of the present invention preferably comprise a safe and effective amount of the branched primary alcohol component described herein. For example, the personal care compositions of the present invention may comprise from about 0.01 to about 30%, preferably from about 0.1 to about 20%, more preferably from about 0.5% to about 15% and especially from about 1% to about 10% by weight of the branched primary alcohol component.

As used herein, the phrase “average number of branches per molecule chain” refers to the average number of branches per alcohol molecule, as measured by ¹³C Nuclear Magnetic Resonance (¹³C NMR) as discussed below, or optionally ¹H Proton NMR. The average number of carbon atoms in the chain is determined by gas chromatography with a mass selective detector.

Various references will be made throughout this specification and the claims to the percentage of branching at a given carbon position, the percentage of branching based on types of branches, average number of branches, and percentage of quaternary atoms. These amounts are to be measured and determined by using a combination of the following three ¹³C-NMR techniques. (1) The first is the standard inverse gated technique using a 45-degree tip ¹³C pulse and 10 s recycle delay (an organic free radical relaxation agent is added to the solution of the branched alcohol in deuterated chloroform to ensure quantitative results). (2) The second is a J-Modulated Spin Echo NMR technique (JMSE) using a 1/J delay of 8 ms (J is the 125 Hz coupling constant between carbon and proton for these aliphatic alcohols). This sequence distinguishes carbons with an odd number of protons from those bearing an even number of protons, i.e. CH₃/CH vs CH₂/Cq (Cq refers to a quaternary carbon). (3) The third is the JMSE NMR “quat-only” technique using a 1/2J delay of 4 ms which yields a spectrum that contains signals from quaternary carbons only. The JSME NMR quat-only technique for detecting quaternary carbon atoms is sensitive enough to detect the presence of as little as 0.3 atom % of quaternary carbon atoms. As an optional further step, if one desires to confirm a conclusion reached from the results of a quat-only JSME NMR spectrum, one may also run a DEPT-135 NMR sequence. We have found that the DEPT-135 NMR sequence is very helpful in differentiating true quaternary carbons from break-through protonated carbons. This is due to the fact that the DEPT-135 sequence produces the “opposite” spectrum to that of the JMSE “quat-only” experiment. Whereas the latter nulls all signals except for quaternary carbons, the DEPT-135 nulls exclusively quaternary carbons. The combination of the two spectra is therefore very useful in spotting non quaternary carbons in the JMSE “quat-only” spectrum. When referring to the presence or absence of quaternary carbon atoms throughout this specification, however, we mean that the given amount or absence of the quaternary carbon is as measured by the quat-only JSME NMR method. If one optionally desires to confirm the results, then one may also use the DEPT-135 technique to confirm the presence and amount of a quaternary carbon.

The primary alcohol component used in one embodiment of the invention contains an average chain length per molecule ranging from about 8 to about 36 carbon atoms, preferably from about 11 to about 21 carbon atoms. The number of carbon atoms includes carbon atoms along the chain backbone as well as branching carbons, but does not include carbon atoms in alkylene oxide groups.

Preferably, at least about 75 wt %, more preferably, at least about 90 wt. % of the molecules in the primary alcohol component have chain lengths ranging from about 11 to about 21, yet more preferably from about 14 to about 18 carbon atoms.

In one embodiment of the invention, the average number of branches per molecule is at least about 0.7, as defined and determined above. Preferred alcohol components are those having an average number of branches of from about 0.7 to about 3.0, preferably from about 1.0 to about 3.0. Particularly preferred alcohol components are those having an average number of branches of at least about 1.5, in particular ranging from about 1.5 to about 2.3, especially from about 1.7 to about 2.1.

In a preferred embodiment of the invention the primary alcohol component has less than about 0.5 atom % of Cq's as measured by a quat-only JMSE modified 13C-NMR having a detection limit of 0.3 atom % or better, and preferably contains no Cq's as measured by this NMR technique. For reasons not yet clearly understood, it is believed that the presence of Cq's on an alcohol molecule prevents the biodegradation by biological organisms. Alcohols containing as little as 1 atom % of Cq's have been been found to biodegrade at failure rates.

In a preferred embodiment of the invention, less than about 5%, or more preferably less than about 3%, of the alcohol molecules in the primary alcohol component are linear alcohols. The efficient reduction in the number of linear alcohols to such a small amount in the composition results from introducing branching on an olefin feedstock either by a skeletal isomerization or a dimerisation technique using efficient catalysts as described further below, rather than introducing branching by methods such as acid catalyzed oligomerization of propylene molecules, or zeolite catalyzed oligomerization techniques. The percentage of molecules which are linear may be determined by gas chromatography.

### Skeletal Isomerization

In a preferred embodiment herein, the branching is introduced by skeletal isomerization.

When the branching has been achieved by skeletal isomerization, the primary alcohol component used herein may be characterized by the NMR technique as having from about 5 to about 25% branching on the C2 carbon position, relative to the hydroxyl carbon atom. In a more preferred embodiment, from about 10 to about 20% of the number of branches are at the C2 position, as determined by the NMR technique. In these embodiments, the primary alcohol component may also generally have from about 10% to about 50% of the number of branches on the C3 position, more typically from about 15% to about 30% on the C3 position, also as determined by the NMR technique. When coupled with the number of branches seen at the C2 position, the primary alcohol component in these embodiments contains significant amount of branching at the C2 and C3 carbon positions.

Not only does the primary alcohol component used in these embodiments of the present invention have a significant number of branches at the C2 and C3 positions, but as may be seen by the NMR technique, many of the primary alcohol components have at least about 5% of isopropyl terminal type of branching, meaning methyl branches at the second to last carbon position in the backbone relative to the hydroxyl carbon. Even at least about 10% of terminal isopropyl types of branches in the primary alcohol component of these embodiments, typically in the range of about 10% to about 20%, may be seen. In typical hydroformylated olefins of the NEODOL series commercially available from The Shell Chemical Company, less than 1%, and usually 0.0%, of the branches are terminal isopropyl branches. By skeletally isomerizing the olefin according to these embodiments of the invention, however, the primary alcohol component contains a high percentage of terminal isopropyl branches relative to the total number of branches.

Considering the combined number of branches occurring at the C2, C3, and isopropyl positions, there are embodiments of the invention where at least about 20%, more preferably at least about 30%, of the branches are concentrated at these positions. The scope of the invention, however, includes branching occurring across the length of the carbon backbone.

The types of branching found in the primary alcohol composition of the invention varies from methyl, ethyl, propyl, and butyl or higher.

In a preferred embodiment of the invention, the total number of methyl branches number at least about 40%, even at least about 50%, of the total number of branches, as measured by the NMR technique described above. This percentage includes the overall number of methyl branches seen by the NMR technique described above within the C1 to the C3 carbon positions relative to the hydroxyl group, and the terminal isopropyl type of methyl branches.

The primary alcohol component of some embodiments herein contains a significant increase in the number of ethyl branches-over those seen on NEODOL alcohols such as NEODOL 45. For example, in some embodiments the number of ethyl branches can range from about 5% to about 30%, most typically from about 10% to about 20%, based on the overall types of branching that the NMR method detects. Thus, the skeletal isomerization of the olefins produces both methyl and ethyl branches. Thus, the types of catalysts one may use to perform skeletal isomerization are not restricted to those which will produce only methyl branches. The presence of a variety of branching types may enhance a good overall balance of properties.

The olefins used in the olefin feed for skeletal isomerization are preferably at least C₇ mono-olefins. In a preferred range, the olefin feed comprises C₇ to C₃₅ mono-olefins. Olefins in the C₁₁ to C₁₉ range are considered most preferred for use herein, to produce primary alcohol components in the C₁₂ to C₂₀ range.

In general, the olefins in the olefin feed composition are preferably predominantly linear. Attempting to process a predominantly branched olefin feed, containing quaternary carbon atoms or extremely high branch lengths, would require separation methods after passing the olefin stream across the catalyst bed to separate these species from the desired branched olefins. While the olefin feed can contain some branched olefins, the olefin feed processed for skeletal isomerization preferably contains greater than about 50 percent, more preferably greater than about 70 percent, and most preferably greater than about 80 mole percent or more of linear olefin molecules.

The olefin feed generally does not consist of 100% olefins within the specified carbon number range, as such purity is not commercially available. The olefin feed is usually a distribution of mono-olefins having different carbon lengths, with at least 50 wt. % of the olefins being within the stated carbon chain range or digit, however specified. Preferably, the olefin feed will contain greater than 70 wt. %, more preferably about 80 wt. % or more of mono-olefins in a specified carbon number range (e.g., C₇ to C₉, C₁₀ to C₁₂, C₁₁ to C₁₅, C₁₂ to C₁₃, C₁₅ to C₁₈, etc.), the remainder of the product being olefin of other carbon number or carbon structure, diolefins, paraffins, aromatics, and other impurities resulting from the synthesis process. The location of the double bond is not limited. The olefin feed composition may comprise α-olefins, internal olefins, or a mixture thereof.

Chevron Alpha Olefin product series (trademark of and sold by Chevron Chemical Co.), manufactures predominantly linear olefins by the cracking of paraffin wax. Commercial olefin products manufactured by ethylene oligomerization are marketed in the United States by Shell Chemical Company under the trademark NEODENE and by Ethyl Corporation as Ethyl Alpha-Olefins. Specific procedures for preparing suitable linear olefins from ethylene are described in U.S. Pat. Nos. 3,676,523, 3,686,351, 3,737,475, 3,825,615 and 4,020,121. While most of such olefin products are comprised largely of alpha-olefins, higher linear internal olefins are also commercially produced, for example, by the chlorination-dehydro-chlorination of paraffins, by paraffin dehydrogenation, and by isomerization of alpha-olefins. Linear internal olefin products in the C₈ to C₂₂ range are marketed by Shell Chemical Company and by Liquichemica Company.

Skeletal isomerisation of linear olefins may be carried out by any known means. Preferably herein, skeletal isomerisation is carried out using the process of U.S. Pat. No. 5,849,960, with use of a catalytic isomerisation furnace. Preferably an isomerisation feed as hereinbefore defined is contacted with an isomerisation catalyst which is effective for skeletal isomerising a linear olefin composition into an olefin composition having an average number of branches per molecule chain of at least about 0.7. More preferably the catalyst comprises a zeolite having at least one channel with a crystallographic free channel diameter ranging from greater than 4.2 Angstrom and less than 7 Angstrom, measured at room temperature, with essentially no channel present which has a free channel diameter which is greater than 7 Angstrom.

Suitable zeolites are described in U.S. Pat. No. 5,510,306, the contents of which are incorporated herein by reference, and are described in the Atlas of Zeolite Structure Types by W. M. Meier and D. H. Olson. Preferred catalysts include ferrierite, A1PO-31, SAPO-11, SAPO-31, SAPO-41, FU-9, NU-10, NU-23, ZSM-12, ZSM-22, ZSM-23, ZSM-35, ZSM-48, ZSM-50, ZSM-57, SUZ-4A, MeAPO-11, MeAPO-31, MeAPO-41, MeAPSO-11, MeAPSO-31, and MeAPSO-41, MeAPSO-46, ELAPO-11, ELAPO-31, ELAPO-41, ELAPSO-11, ELAPSO-31, and ELAPSO-41, laumontite, cancrinite, offretite, hydrogen form of stilbite, the magnesium or calcium form of mordenite and partheite, and their isotypic structures. Combinations of zeolites can also be used herein. These combinations can include pellets of mixed zeolites and stacked bed arrangements of catalyst such as, for example, ZSM-22 and/or ZSM-23 over ferrierite, ferrierite over ZSM-22 and/or ZSM-23, and ZSM-22 over ZSM-23. The stacked catalysts can be of the same shape and/or size or of different shape and/or size such as ⅛ inch trilobes over 1/32 inch cylinders for example. Alternatively natural zeolites may be altered by ion exchange processes to remove or substitute the alkali or alkaline earth metal, thereby introducing larger channel sizes or reducing larger channel sizes. Such zeolites include natural and synthetic ferrierite (can be orthorhombic or monoclinic), Sr-D, FU-9 (EP B-55,529), ISI-6 (U.S. Pat. No. 4,578,259), NU-23 (E.P.A.-103,981), ZSM-35 (U.S. Pat. No. 4,016,245) and ZSM-38 (U.S. Pat. No. 4,375,573). Most preferably the catalyst is ferrierite.

The skeletal isomerisation catalyst is suitably combined with a refractory oxide as binding material in known manner, for example natural clays, such as bentonite, montmorillonite, attapulgite, and kaolin; alumina; silica; silica-alumina; hydrated alumina; titania; zirconia and mixtures thereof. More preferred binders are aluminas, such as pseudoboehmite, gamma and bayerite aluminas. These binders are readily available commercially and are used to manufacture alumina-based catalysts.

The weight ratio of zeolite to binder material suitably ranges from about 10:90 to about 99.5:0.5, preferably from about 75:25 to about 99:1, more preferably from about 80:20 to about 98:2 and most preferably from about 85:15 to about 95:5 (anhydrous basis).

Preferably, the skeletal isomerization catalyst is also prepared with at least one acid selected from mono-carboxylic acids and inorganic acids and at least one organic acid with at least two carboxylic acid groups (“polycarboxylic acid”). Suitable acids include those disclosed in U.S. Pat. No. 5,849,960.

Optionally, coke oxidation promoting metals can be incorporated into the instant catalysts to promote the oxidation of coke in the presence of oxygen at a temperature greater than about 250° C. Suitable coke oxidation promoting materials include those disclosed in U.S. Pat. No. 5,849,960.

In a preferred method, the instant catalysts can be prepared by mixing a mixture of at least one zeolite as herein defined, alumina-containing binder, water, at least one monocarboxylic acid or inorganic acid and at least one polycarboxylic acid in a vessel or a container, forming a pellet of the mixed mixture and calcining the pellets at elevated temperatures. Preparation methods of the catalyst are described in U.S. Pat. No. 5,849,960.

High conversion, high selectivity, and high yields are attained by the process described herein.

The present skeletal isomerization process can be operated at a wide range of conditions. Preferably skeletal isomerisation is conducted at elevated temperature in the range 200° C. to 500° C., more preferably 250 to 350° C., and at pressure ranging from 0.1 atmospheres (10 kPa) to 10 atmospheres (1 MPa), more preferably from 0.5 to 5 atmospheres (50 to 500 kPa). Olefin weight hour space velocity (WHSV) can range from 0.1 to 100 per hour. Preferably, the WHSV is between 0.5 to 50, more preferably between 1 and 40, most preferably between 2 and 30 per hour. At lower WHSV's, it is possible to operate at lower temperatures while achieving high yields of skeletally isomerized branched olefins. At higher WHSV's, the temperature is generally increased in order to maintain the desired conversion and selectivity to the skeletally isomerized branched olefins. Further, optimal selectivities are generally achieved at lower olefin partial pressures mentioned above. For this reason, it is often advantageous to dilute the feed stream with a diluent gas such as nitrogen or hydrogen. Although reducing the olefin partial pressure with a diluent may be beneficial to improve the selectivity of the process, it is not necessary to dilute the olefin stream with a diluent.

If a diluent is used, the molar ratio of olefin to diluent can range from 0.01:1 to 100:1, and is generally within the range of 0.1:1 to 5:1.

Although in the present invention, skeletal isomerization is preferred, dimerization is yet another option for achieving branching.

Broadly speaking, a primary alcohol component is obtained by dimerizing an olefin feed comprising C6-C10 linear olefins in the presence of a dimerization catalyst under dimerization conditions to obtain C12-C20 olefins. Details of suitable dimerisation processes, including process conditions, olefin feed and suitable catalysts, are to be found in U.S. Pat. No. 5,780,694.

### Hydroformylation

The branched, preferably skeletally isomerized or dimerized, olefins may be subsequently converted to a primary alcohol component, for example, by hydroformylation. For example, in hydroformylation, skeletally isomerized olefins may be converted to alkanols by reaction with carbon monoxide and hydrogen according to the Oxo process. Most commonly used is the “modified Oxo process”, using a phosphine, phosphite, arsine or pyridine ligand modified cobalt or rhodium catalyst, as described in U.S. Pat. Nos. 3,231,621; 3,239,566; 3,239,569; 3,239,570; 3,239,571; 3,420,898; 3,440,291; 3,448,158; 3,448,157; 3,496,203; and 3,496,204; 3,501,515; and 3,527,818. Methods of production are also described in Kirk Othmer, “Encyclopedia of Chemical Technology” 3rd Ed. vol 16, pages 637-653; “Monohydric Alcohols: Manufacture, Applications and Chemistry”, E. J. Wickson, Ed. Am. Chem. Soc. 1981.

Hydroformylation is a term used in the art to denote the reaction of an olefin with CO and H2 to produce an aldehyde/alcohol which has one more carbon atom than the reactant olefin. Frequently, in the art, the term hydroformylation is utilized to cover the aldehyde and the reduction to the alcohol step in total, i.e., hydroformylation refers to the production of alcohols from olefins via carbonylation and an aldehyde reduction process. As used herein, hydroformylation refers to the ultimate production of alcohols.

Illustrative catalysts include, but are not necessarily limited to, cobalt hydrocarbonyl catalysts and metal-phosphine ligand catalysts comprising metals, including but not limited to, palladium, cobalt and rhodium. The choice of catalysts determines the various reaction conditions imposed. These conditions can vary widely, depending upon the particular catalysts. For example, temperatures can range from about room temperatures to 300° C. When cobalt carbonyl catalysts are used, which are also the ones typically used, temperatures will range from 150° to 250° C. One of ordinary skill in the art, by referring to the above-cited references, or any of the well-known literature on oxo alcohols can readily determine those conditions of temperature and pressure that will be needed to hydroformylate the isomerized or dimerized olefins.

Typical reaction conditions, however, are moderate. Temperatures in the range of 125° C. to 200° C. are recommended. Reaction pressures in the range of 2170 to 10440 kPa are typical, but lower or higher pressures may be selected. Ratios of catalyst to olefin ranging from 1:1000 to 1:1 are suitable. The ratio of hydrogen to carbon monoxide can vary widely, but is usually in the range of 1 to 10, preferably about 2 moles of hydrogen to one mole of carbon monoxide to favor the alcohol product.

The hydroformylation process can be carried out in the presence of an inert solvent, although it is not necessary. A variety of solvents can be applied such as ketones, e.g. acetone, methyl ethyl ketone, methyl iso-butyl ketone, acetophenone and cyclohexanone; aromatic compounds such as benzene, toluene and the xylenes; halogenated aromatic compounds such as chlorobenzene and orthodichlorobenzene; halogenated paraffinic hydrocarbons such as methylene chloride and carbon tetrachloride; paraffins such as hexane, heptane, methylcyclohexane and isooctane and nitriles such as benzonitrile and acetonitrile.

With respect to the catalyst ligand, mention may be made of tertiary organo phosphines, such as trialkyl phosphines, triamyl phosphine, trihexyl phosphine, dimethyl ethyl phosphine, diamylethyl phosphine, tricyclopentyl(or hexyl) phosphine, diphenyl butyl phosphine, diphenyl benzyl phosphine, triethoxy phosphine, butyl diethyoxy phosphine, triphenyl phosphine, dimethyl phenyl phosphine, methyl diphenyl phosphine, dimethyl propyl phosphine, the tritolyl phosphines and the corresponding arsines and stibines.

Included as bidentate-type ligands are tetramethyl diphosphinoethane, tetramethyl diphosphinopropane, tetraethyl diphosphinoethane, tetrabutyl diphosphinoethane, dimethyl diethyl diphosphinoethane, tetraphenyl diphosphinoethane, tetraperfluorophenyl diphosphinoethane, tetraphenyl diphosphinopropane, tetraphenyl diphosphinobutane, dimethyl diphenyl diphosphinoethane, diethyl diphenyl diphosphinopropane and tetratrolyl diphosphinoethane.

Examples of other suitable ligands are the phosphabicyclohydrocarbons, such as 9-hydrocarbyl-9-phosphabicyclononane in which the smallest P-containing ring contains at least 5 carbon atoms. Some examples include 9-aryl-9-phosphabicyclo[4.2.1]nonane, (di)alkyl-9-aryl-9-phosphabicyclo[4.2.1]nonane, 9-alkyl-9-phosphabi-cyclo[4.2.1]nonane, 9-cycloalkyl-9-phosphabicyclo-[4.2.1]nonane, 9-cycloalkenyl-9-phosphabicyclo-[4.2.1]nonane, and their [3.3.1] and [3.2.1] counter-parts, as well as their triene counterparts.

### Ethoxylation

As mentioned above, the branched primary alcohol component may optionally comprise up to about 3 moles of alkylene oxide per mole of alcohol. The upper limit on the number of moles of alkylene oxide reflects a preference that the primary alcohol component not act as a surfactant in the compositions herein.

Suitable oxyalkylated alcohols may be prepared by adding to the alcohol or mixture of alcohols to be oxyalkylated a calculated amount, e.g., from about 0.1% by weight to about 0.6% by weight, preferably from about 0.1% by weight to about 0.4% by weight, based on total alcohol, of a strong base, typically an alkali metal or alkaline earth metal hydroxide such as sodium hydroxide or potassium hydroxide, which serves as a catalyst for oxyalkylation. The resulting mixture is dried, as by vapour phase removal of any water present, and an amount of alkylene oxide calculated to provide from about 1 mole to about 3 moles of alkylene oxide per mole of alcohol is then introduced and the resulting mixture is allowed to react until the alkylene oxide is consumed, the course of the reaction being followed by the decrease in reaction pressure.

Further details of suitable oxyalkylation processes including process conditions can be found in U.S. Pat. No. 6,150,322.

Examples of suitable alkylene oxides for use herein include ethylene oxide, propylene oxide and butylene oxide, and mixtures thereof, preferably ethylene oxide.

### Cosmetically-Acceptable Vehicle

The personal care compositions herein also comprise a cosmetically-acceptable vehicle in addition to the primary branched alcohol component. Preferably, the cosmetically-acceptable vehicle is generally present in a safe and effective amount, preferably from about 1% to about 99.99%, more preferably from about 20% to about 99%, especially from about 60% to about 90%. The cosmetically-acceptable vehicle may contain a variety of components suitable for rendering such compositions cosmetically, aesthetically or otherwise, acceptable or to provide them with additional usage benefits. The components of the cosmetically-acceptable vehicle preferably should be physically and chemically compatible with the primary branched alcohol component and preferably should not unduly impair the stability, efficacy or other benefits associated with the personal care compositions of the invention.

Suitable ingredients for inclusion in the cosmetically-acceptable vehicle include, but are not limited to, emollients, oil absorbents, antimicrobial agents, binders, buffering agents, denaturants, cosmetic astringents, film formers, humectants, surfactants, emulsifiers, sunscreen agents, oils such as vegetable oils, mineral oil and silicone oils, opacifying agents, perfumes, colouring agents, pigments, skin soothing and healing agents, preservatives, propellants, skin penetration enhancers, solvents, suspending agents, emulsifiers, cleansing agents, thickening agents, solubilising agents, waxes, inorganic sunblocks, sunless tanning agents, antioxidants and/or free radical scavengers, chelating agents, suspending agents, sunless tanning agents, antioxidants and/or radical scavengers, anti-acne agents, anti-dandruff agents, anti-inflammatory agents, exfolients/desquamation agents, organic hydroxy acids, vitamins, natural extracts, inorganic particulates such as silica and boron nitride, deodorants and antiperspirants.

Non-limiting examples of such materials are described in Harry's Cosmeticology, 7^{th} Edition., Harry & Wilkinson (Hill Publishers, London 1982); in The Chemistry and Manufacture of Cosmetics, 2^{nd}. Edition., deNavarre (Van Nostrand 1962-1965); and in the Handbook of Cosmetic Science and Technology, 1^{st} Edition., Knowlton & Pearce (Elsevier 1993); CTFA International Cosmetic Ingredient Dictionary and Handbook, 7^{th} Edition, volume 2, edited by Wenniger and McEwen (The Cosmetic, Toiletry, and Fragrance Association, Inc., Washington, D.C., 1997); and WO01/89466.

Preferred compositions have an apparent viscosity of from about 5,000 to about 2,000,000 mPa.s, measured using a Brookfield DVII RV viscometer, spindle TD, at 5 rpm, 25° C. and ambient pressure. The viscosity will vary depending on whether the composition is a cream or lotion.

Compositions of the present invention are preferably aqueous, and more preferably are in the form of an emulsion, such as an oil-in-water or water-in-oil emulsion. For example, in the case of an oil-in-water emulsion a hydrophobic phase containing an oily material is dispersed within an aqueous phase. Oil-in-water emulsions typically comprise from about 1% to about 50%, preferably from about 1% to about 30% by weight of the dispersed hydrophobic phase and from about 1% to about 99%, more preferably from about 40% to about 90% by weight of the continuous aqueous phase. The emulsion may also comprise a gel network, such as described in G. M. Eccelston, Application of Emulsion Stability Theories to Mobile and Semisolid O/W Emulsions, Cosmetic & Toiletries, Vol. 101, November 1996, pp. 73-92.

The compositions of the invention will preferably be formulated to have a pH of from about 4.5 to about 9, more preferably from about 5 to about 8.5.

The compositions herein can be formulated into a wide variety of product forms such as are known in the art and can be used for a wide variety of purposes. Suitable product forms include, but are not limited to, lotions, creams, gels, sticks, sprays, ointments, pastes and mousses.

The compositions of the present invention can be formulated into either non-cleansing or cleansing formulations. Examples of non-cleansing formulations include hair conditioners, skin moisturizing creams, suncreen compositions, night creams, antiperspirants, lipsticks, cosmetic foundations, body lotions, and the like. Examples of cleansing formulations include shampoos, facial cleansers, shower gels, bath foams, hand cleansers, and the like. Generally, cleansing formulations contain relatively high levels of surfactants, generally greater than 5%, preferably greater than 10%.

In preferred embodiments herein the personal care compositions are formulated as non-cleansing formulations, preferably comprising about 5% or less, more preferably about 3% of less, by weight, of surfactant.

Any surfactant known for use in personal care compositions can be used herein, provided that the selected agent is chemically and physically compatible with other ingredients in the composition. Suitable surfactants for use in the compositions herein include nonionic, anionic, amphoteric, zwitterionic and cationic surfactants, such as those described in WO01/89466.

Preferred cosmetically-acceptable vehicles herein contain a hydrophilic diluent, typically at a level of about 60% to about 99% by weight of composition. Examples of suitable hydrophilic diluents include water, low molecular weight monohydric alcohols, glycols and polyols, including propylene glycol, polypropylene glycol, glycerol, butylene glycol, sorbitol esters, ethanol, isopropanol, ethoxylated ethers, propoxylated ethers and mixtures thereof. A preferred diluent is water.

The cosmetically-acceptable vehicle herein may contain an emulsifier to help disperse and suspend the discontinuous phase within the continuous aqueous phase. An example of a suitable emulsifier is PEG-30 dihydroxystearate commercially available from Uniquema Americas and a mixture of glyceryl stearate and PEG-100 stearate commercially available under the tradename Lipomulse 165 from Lipo Chemicals, Inc.

Preferred compositions herein comprise emollient materials, in addition to the primary branched alcohol component which itself has emolliency properties. Emollients are materials which lubricate the skin, increase the softness and smoothness of the skin, prevent or relieve dryness, and/or protect the skin. Emollients are typically oily or waxy materials which are water-immiscible. In an oil-in-water emulsion, emollients therefore generally form part of the disperse oil phase. Suitable emollients are described in Sagarin, Cosmetics, Science and Technology, 2^{nd} Edition, Vol. 1, pp. 32-43 (1972) and in WO01/89466.

Examples of preferred emollients include those disclosed in WO01/89466 such as straight and branched chain hydrocarbons having from about 7 to about 40 carbon atoms, such as dodecane, squalane, cholesterol, isohexadecane and the C₇-C₄₀ isoparaffins, C₁-C₃₀ alcohol esters of C₁-C₃₀ carboxylic acids and of C₂-C₃₀ dicarboxylic acids such as isononyl isononanoate, isopropyl myristate, myristyl propionate, isopropyl stearate, isopropyl isostearate, methyl isostearate, behenyl behenate, octyl palmitate, dioctyl maleate, diisopropyl adipate, and diisopropyl dilinoleate, C₁-C₃₀ mono- and poly-esters of sugars and related materials such as those disclosed in WO01/89466; and vegetable oils and hydrogenated vegetable oils including safflower oil, castor oil, coconut oil, cottonseed oil, palm kernal oil, palm oil, peanut oil, soybean oil, rapeseed oil, linseed oil, rice bran oil, pine oil, sesame oil, sunflower seed oil, partially and fully hydrogenated oils of the above, and mixtures thereof.

Preferred compositions herein contain silicone-based ingredients such as volatile or non-volatile organopolysiloxane oils. Preferred for use herein are organopolysiloxanes selected from polyalkylsiloxanes, alkyl substituted dimethicones, dimethiconols, polyalkylaryl siloxanes and cyclomethicones, preferably polyalkylsiloxanes and cyclomethicones. Also useful herein are silicone-based emulisifers such as dimethicone copolyols, an example of which is cetyl dimethicone copolyol, supplied by Goldschmidt under the tradename Abil EM90.

The compositions herein preferably comprise a thickening agent such as those described in WO01/89466. Examples of suitable thickening agents include carboxylic acid polymers, crosslinked polacrylates, polyacrylamides, xanthan gum, cellulose derivatives, and mixtures thereof. Examples of suitable thickening agents include the Carbopol series of materials commercially available from B.F. Goodrich and cetyl hydroxymethyl cellulose supplied by Hercules Aqualon under the tradename Natrosol 250 HR CS.

Preferred compositions herein comprise a humectant at a level of about 5% to about 30% by weight. Preferred humectants include, but are not limited to, glycerine, polyoxyalkylene gycol, urea, D or DL panthenol and alkylene glycols such as propylene glycol or butylene glycol.

When it is desired to provide protection from the harmful effects of the sun, the compositions herein may contain a safe and effective amount of one or more sunscreen ingredients, selected from inorganic or organic sunscreens. Examples of suitable sunscreens include those disclosed in WO01/89466.

The compositions herein may comprise a long-chain alcohol in addition to the branched primary alcohol component. Suitable long-chain alcohols may be selected from, for example, linear or branched, saturated or unsaturated alcohols having an average number of carbon atoms in the range of from about 8 to about 36.

Examples of naturally derived long-chain alcohols include the fatty alcohols cetyl alcohol, stearyl alcohol and behenyl alcohol.

Other examples of suitable long-chain alcohols include those commercially available from The Shell Chemical Company under the tradename NEODOL. Examples of NEODOL alcohols include NEODOL 23, NEODOL 91, NEODOL 1, NEODOL 45 and NEODOL 25. All of these alcohols are predominantly linear alcohols.

Other examples of suitable alcohols include alcohols of the SAFOL series such as SAFOL 23, alcohols of the LIAL series such LIAL 123, and alcohols of the ALFONIC series, all of which are commercially available from Sasol.

Also suitable for use herein are, for example, the so-called “Guerbet” alcohols, for example, EUTANOL G16, commercially available from Cognis Corporation.

The compositions herein may be prepared according to procedures usually used in cosmetics and that are well known and understood by those skilled in the art.

The following examples will illustrate the nature of the invention, but are not intending to be limiting in any way.

### EXAMPLE 1

This example will demonstrate the manufacture of a skeletally isomerized C₁₆ olefin, subsequently converted to a skeletally isomerized C₁₇ primary alcohol component. The manufacturing process for this Example is as described in Example 1 of U.S. Pat. No. 5,849,960, but is reproduced here for convenience.

1 Litre of NEODENE 16 olefin, a C₁₆ linear α-olefin commercially available from Shell Chemical Company, was first dried and purified through alumina. The olefin was then passed through a tube furnace at about 250° C. set at, a feed rate of about 1.0 ml/minute and using a nitrogen pad flowing at about 91 ml/minute. Working from the top, the tube furnace was loaded with glass wool, then 10 ml of silicon carbide, then the catalyst, followed by 5 ml of silicon carbide, and more glass wool at the bottom. The volume of the tube furnace was 66 ml. The reactor tube furnace had three temperature zones, with a multi-point thermocouple inserted into the tube reactor and positioned such that the temperature above, below and at three different places in the catalyst bed could be monitored. The reactor was inverted and installed in the furnace. All three zones, including the catalyst zone, were kept at about 250° C. during the reaction and the pressure was maintained in the reactor at 114 kPa. The amount of catalyst used was 23.1 g, or 53 ml by volume. The type of catalyst used to structurally isomerize the NEODENE 16 olefin was a 1.59 mm extruded and calcined H-ferrierite containing 100 ppm palladium metal.

This catalyst was prepared in accordance with example C of U.S. Pat. No. 5,510,306, reproduced in part herein for convenience. An ammonium-ferrierite having a molar silica to alumina ratio of 62:1, a surface area of 369 square meters per gram (P/Po=0.03), a soda content of 480 ppm and n-hexane sorption capacity of 7.3 g per 100 g of zeolite was used as the starting zeolite. The catalyst components were mulled using a Lancaster mix muller. The mulled catalyst material was extruded using an 25.4 mm or a 57.2 mm Bonnot pin barrel extruder.

The catalyst was prepared using 1 wt % acetic acid and 1 wt % citric acid. The Lancaster mix muller was loaded with 645 grams of ammonium-ferrierite (5.4% Loss on Ignition) and 91 grams of CATAPAL D alumina (LOI of 25.7%). The alumina was blended with the ferrierite for 5 minutes during which time 152 millilitres of deionized water was added. A mixture of 6.8 grams glacial acetic acid, 7.0 grams of citric acid and 152 milliliters of deionized water was added slowly to the muller in order to peptize the alumina. The mixture was mulled for 10 minutes. 0.20 grams of tetra-ammine palladium nitrate in 153 grams of deionized water were then added slowly as the mixture was mulled for a period of 5 additional minutes. Ten grams of METHOCEL F4M hydroxypropyl methylcellulose was added and the zeolite/alumina mixture was mulled for 15 additional minutes. The extrusion mix had an LOI of 43.5%. The 90:10 zeolite/alumina mixture was transferred to the 2.25 inch Bonnot extruder and extruded using a die plate with 1.59 mm holes.

The moist extrudates were tray dried in an oven heated to 150° C. for 2 hours, and then increased to 175° C. for 4 hours. After drying, the extrudates were longs-broken manually. The extrudates were calcined in flowing air at 500° C. for two hours.

The olefin was passed through the reactor furnace over a 5 hour period. Samples of 36.99 g and 185.38 g were collected at about the 1 and 5 hour point, and combined for a total of about 222 g. A portion of this sample was then vacuum distilled at 0.533 kPa to obtain a predominate amount of the C₁₆ skeletally isomerized olefin by collecting distillate cuts boiling at 160° C. in the pot and 85° C. at the head, and 182° C. in the pot and 75° C. at the head.

A 90 gram sample of the 110.93 grams of the skeletally isomerized olefin was then hydroformlyated using the modified oxo process. 90 grams of the skeletally isomerized olefin was reacted with hydrogen and carbon monoxide in about a 1.7:1 molar ratio in the presence of a phosphine modified cobalt catalyst at a temperature of up to about 185° C. and a pressure of about 7684 kPa for 4.5 hours in a nitrogen-purged 300 cc autoclave. After completion of the reaction, the product was cooled to 60° C.

40 grams of the hydroformylated product was poured into a 100 ml flask and vacuum distilled for 4 hours at 0.533 kPa with temperature increases from a start temperature of 89° C. to a finish temperature of 165° C. Distillate cuts of 20.14 g and 4.12 g were taken at 155° C. and 165° C., respectively, and combined in a 100 ml flask.

To the distillate cuts in the flask was added 0.2 g of sodium borohydride, stirred, and heated up to 90° C. over an 8 hour period to deactivate the hydroformylation catalyst and stabilize the alcohols. The distilled alcohol was washed with 90° C. water three times, dried with sodium sulfate, and filtered into a 100 ml flask. The alcohol was then vacuum distilled for a further hour to distill off any remaining water.

The primary alcohol component of Example 1 was subsequently tested for amount, type, and location of branching using the JSME NMR method described herein. For a determination of quaternary carbon atoms, the quat-only JSME NMR technique described herein was used.

Results were as follows: The average number of carbon atoms in the primary alcohol component prepared according to Example 1 was found to be 17, with an average of 1.6 branches per chain. 67.9% of branching occurred at the C4 position and further (relative to the hydroxyl carbon), with 21% of branching at C3, 4% of methyl branching at C2, 1.2% of ethyl branching at C2, 5.9% of propyl branching and longer at C2, 41.7% propyl branching and longer, 16.3% ethyl branching and longer, 42% methyl branching, 0% isopropyl terminal branching, <1% linear alcohol.

Finally, in spite of the high number of branches per molecule chain, no quaternary carbon atoms were detected by the modified NMR JSME method. This would suggest that the compounds of Example 1 should readily biodegrade.

### FORMULATION EXAMPLES

### EXAMPLE 2

### Night Cream (Water-in-oil Emulsion)

To prepare the night cream of Example 2 below, the ingredients of phase A are combined at 75° C., the ingredients of phase B are combined at 50° C. and then phase B is slowly added to phase A. The two phases are mixed until a homogeneous mixture results.

| Phase | Ingredient | Wt % |
|---|---|---|
| A | Abil EM90¹ | 5 |
| A | Arlacel P135² | 1 |
| A | Castorwax MP70³ | 2.5 |
| A | Octyl Palmitate | 5 |
| A | Alcohol component* | 15 |
| A | Vitamin E acetate | 0.1 |
| B | Propylene glycol | 2.5 |
| B | Natrosol 250HR CS⁴ | 0.8 |
| B | Sodium chloride | 0.75 |
| B | Glydant⁵ | 0.2 |
| B | Deionized Water | to 100 |

¹Cetyl Dimethicone Copolyol supplied by Goldschmidt
²PEG-30 Dihydroxystearate supplied by Uniqema Americas
³Hydrogenated Castor Oil supplied by CasChem, Inc.
⁴Cetyl Hydroxymethylcellulose supplied by Hercules/Aqualon
⁵DMDM Hydantoin preservative supplied by Lonza Inc.
*NEODOL 67, a commercially available C₁₆-C₁₇ alcohol from Shell Chemical Company prepared in a manner similar to the C₁₇ alcohol of Example 1

### EXAMPLE 3

### Comparative Example

A Night Cream was prepared in the same way as for Example 2 above except that the alcohol component of Example 2 was replaced by the Guerbet alcohol, Eutanol G16, commercially available from Cognis Corporation. Eutanol G16 has the chemical name 2-hexadecanol, thus has a carbon chain containing 10 carbon atoms with a carbon chain branch containing 6 carbon atoms at the C2 carbon position.

### EXAMPLE 4

### Comparative Example

A Night Cream was prepared in the same way as for Example 2 above except that the branched alcohol component of Example 2 was replaced by, NEODOL 45, which is a mixture of C₁₄ and C₁₅ primarily linear alcohols, commercially available from The Shell Chemical Company.

### EXAMPLE 5

### Moisturiser (Oil-in-water Emulsion)

The moisturizer of Example 5 is prepared by combining the ingredients of phase A at 75° C., combining the ingredients of phase B at 75° C. and adding phase B to phase A. Phase C is added to the resulting mixture and cooled to 40° C. Finally Phase D is added.

| Phase | Ingredient | Wt % |
|---|---|---|
| A | Deionised water | to 100 |
| A | Tetrasodium EDTA | 0.1 |
| A | Glycerine | 2.5 |
| A | Carbopol 980⁶ (2% | 15 |
| | solution) | |
| B | Alcohol component* | 10 |
| B | Lipomulse 165⁷ | 2.5 |
| B | Stearic Acid | 2.5 |
| B | Cetearyl Alcohol | 1 |
| B | Dimethicone DC200- | 1 |
| | 50⁸ | |
| C | NaOH (20% solution) | 0.77 |
| D | Germaben II⁹ | 1 |

⁶Carbomer supplied by B. F. Goodrich
⁷Glyceryl Stearate and PEG 100 Stearate supplied by Lipo Chemicals, Inc.
⁸Supplied by Dow Corning
⁹Propylene Glycol and Diazolidinyl Urea and Methylparaben and Propylparaben preservative supplied by Sutton Laboratories
*NEODOL 67, a commercially available C₁₆-C₁₇ alcohol from Shell Chemical Company prepared in a manner similar to the C₁₇ alcohol of Example 1

The pH of the final formulation was measured to be 6.9.

### EXAMPLE 6

### Comparative Example

A moisturizer was prepared in the same way as Example 5 above except that the branched alcohol component in Example 5 was replaced by Eutanol G16. The pH of the final formulation was measured to be 7.1.

### EXAMPLE 7

### Comparative Example

A moisturizer was prepared in the same way as Example 5 above except that the branched alcohol component in Example 5 was replaced by NEODOL 45. The pH of the final formulation was measured to be 6.3.

### Viscosity Data

The viscosity of each of formulations Examples 2-7 were measured using a Brookfield Viscometer, Spindle No. 5, 20 rpm, room temperature, 1At pressure, unless otherwise specified. The results of these viscosity measurements are shown in Table 1 below.

TABLE 1

| Formulation Example | Viscosity (cps) |
|---|---|
| 2 | 10,400 |
| 3* | 7,600 |
| 4* | 152,000 |
| 5 | 35,600** |
| 6* | 11,300 |
| 7* | 220,000** |

*Comparative Example
**Viscosity settings were Spindle TB, 5 rpm, 1 At pressure, room temperature

The viscosity results show that the compositions containing NEODOL 67, a C₁₆-C₁₇ alcohol prepared in a manner similar to the branched primary alcohol component of Example 1, have a higher viscosity than the compositions containing Eutanol G16 and a lower viscosity than the compositions containing NEODOL 45.

It should be noted however that the formulations containing NEODOL 45 were not as easy to formulate as the formulations containing NEODOL 67, since the NEODOL 67 is liquid at room temperature, whereas NEODOL 45 is supplied in the form of flakes or powder.

All formulation examples were found to have excellent stability.

The results above demonstrate that personal care formulations of the present invention, containing a highly branched primary alcohol component such as that prepared in Example 1, exhibit good stability, excellent viscosity and rheology characteristics and excellent formulation characteristics. These results thus demonstrate that highly branched alcohol components such as those prepared according to Example 1 are useful ingredients for inclusion in personal care compositions.

These results also demonstrate that the compositions of the present invention containing a highly branched alcohol component such as that prepared according to Example 1 display improved characteristics compared to compositions containing the less branched commercially available alcohols, NEODOL 45 and EUTANOL G16. In particular, although the formulations containing the branched alcohol component similar to that of Example 1 have a lower viscosity than the formulations containing NEODOL 45, the former are more suitable as personal care formulations since they are easier to formulate due to the liquid nature of the branched alcohol component.

The branched primary alcohol component prepared in Example 1 and the alcohols used in formulation examples 2 and 5 above may be replaced by any of the branched alcohol components prepared in accordance with Examples 2-5 of U.S. Pat. No. 5,849,960 or Examples 1-3 of U.S. Pat. No. 5,780,694.

What is claimed is:

1. A personal care composition for topical application to the skin or hair comprising
(a) a branched primary alcohol component, having from about 8 to about 36 carbon atoms and an average number of branches per molecule of from about 0.7 to about 3.0, said branching comprising methyl and ethyl branches; and
(b) a cosmetically-acceptable vehicle.

2. The personal care composition of claim 1 wherein the composition comprises about 5% or less by weight of surfactant.

3. The personal care composition of claim 1 wherein the branched primary alcohol component is present in a safe and effective amount.

4. The personal care composition of claim 1 wherein the branched primary alcohol component is present in an amount ranging from about 0.01 to about 30% by weight.

5. The personal care composition of claim 4 wherein the branched primary alcohol component is present in an amount ranging from about 0.1 to about 20% by weight.

6. The personal care composition of claim 5 wherein the branched primary alcohol component is present in an amount ranging from about 0.5% to about 15% by weight.

7. The personal care composition of claim 6 wherein the branched primary alcohol component is present in an amount ranging from about 1% to about 10% by weight.

8. The personal care composition of claim 1 wherein the average chain length per molecule in the branched primary alcohol component ranges from about 8 to about 36 carbon atoms.

9. The personal care composition of claim 8 wherein the average chain length per molecule in the branched primary alcohol component ranges from about 11 to about 21 carbon atoms.

10. The personal care composition of claim 1 wherein the average number of branches per molecule in the branched alcohol component is in the range from about 1.0 to about 3.0.

11. The personal care composition of claim 1 wherein the average number of branches per molecule in the branched alcohol component is at least about 1.5.

12. The personal care composition of claim 10 wherein the average number of branches per molecule in the branched primary alcohol component is in the range from about 1.5 to about 2.3.

13. The personal care composition of claim 12 wherein the average number of branches per molecule in the branched primary alcohol component is in the range from about 1.7 to about 2.1.

14. The personal care composition of claim 1 wherein the branched primary alcohol component comprises less than about 0.5 atom % of quaternary carbon atoms.

15. The personal care composition of claim 1 wherein in the branched primary alcohol component less than about 5% of the alcohol molecules are linear alcohols.

16. The personal care composition of claim 15 wherein in the branched primary alcohol component less than about 3% of the alcohol molecules are linear alcohols.

17. The personal care composition of claim 1 wherein the branched primary alcohol component has from about 5 to about 25% branching on the C2 carbon position, relative to the hydroxyl carbon atom.

18. The personal care composition of claim 17 wherein the branched primary alcohol component has from about 10 to about 20% branching on the C2 carbon position, relative to the hydroxyl carbon atom.

19. The personal care composition of claim 1 wherein the branched primary alcohol component has from about 10% to about 50% branching on the C3 position.

20. The personal care composition of claim 19 wherein the branched primary alcohol component has from about 15% to about 30% branching on the C3 position.

21. The personal care composition of claim 1 wherein the branched primary alcohol component has at least about 5% of isopropyl terminal type of branching.

22. The personal care composition of claim 21 wherein the branched primary alcohol component has at least about 10% of isopropyl terminal type of branching.

23. The personal care composition of claim 22 wherein the branched primary alcohol component has in the range of about 10% to about 20% of isopropyl terminal type of branching.

24. The personal care composition of claim 1 wherein at least about 20% of branches in the branched primary alcohol component occur at the C2, C3, and terminal isopropyl positions.

25. The personal care composition of claim 24 wherein at least about 30% of branches in the branched primary alcohol component occur at the C2, C3, and terminal isopropyl positions.

26. The personal care composition of claim 1 wherein in the branched primary alcohol component at least about 40% of the number of branches in the alcohol are methyl branches.

27. The personal care composition of claim 26 wherein in the branched primary alcohol component at least about 50% of the number of branches in the alcohol are methyl branches.

28. The personal care composition of claim 1 wherein in the branched alcohol component from about 5% to about 30% of the number of branches in the alcohol are ethyl branches.

29. The personal care composition of claim 28 wherein in the branched alcohol component from about 10% to about 20% of the number of branches in the alcohol are ethyl branches.

30. The personal care composition of claim 1 wherein the cosmetically-acceptable vehicle is present in a safe and effective amount.

31. The personal care composition of claim 1 wherein the cosmetically-acceptable vehicle is present in an amount of from about 1% to about 99.99%.

32. The personal care composition of claim 31 wherein the cosmetically-acceptable vehicle is present in an amount of from about 20% to about 99%.

33. The personal care composition of claim 32 wherein the cosmetically-acceptable vehicle is present in an amount of from about 60% to about 90%.

34. The personal care composition of claim 1 wherein the cosmetically-acceptable vehicle comprises an ingredient selected from the group consisting of emollients, oil absorbents, antimicrobial agents, binders, buffering agents, denaturants, cosmetic astringents, film formers, humectants, surfactants, emulsifiers, sunscreen agents, oils, opacifying agents, perfumes, colouring agents, pigments, skin soothing and healing agents, preservatives, propellants, skin penetration enhancers, solvents, suspending agents, emulsifiers, cleansing agents, thickening agents, solubilising agents, waxes, inorganic sunblocks, sunless tanning agents, antioxidants, free radical scavengers, chelating agents, suspending agents, anti-acne agents, anti-dandruff agents, anti-inflammatory agents, exfolients, desquamation agents, organic hydroxy acids, vitamins, natural extracts, inorganic particulates, deodorants, antiperspirants, and mixtures thereof.

35. The personal care composition of claim 34 wherein the cosmetically-acceptable vehicle comprises an oil selected from the group consisting of vegetable oils, mineral oil, silicone oils, and mixtures thereof.

36. The personal care composition of claim 34 wherein the cosmetically-acceptable vehicle comprises an inorganic particulate selected from the group consisting of silica, boron nitride, and mixtures thereof.

37. The personal care composition of claim 1 wherein the cosmetically-acceptable vehicle comprises a surfactant.

38. The personal care composition of claim 1 wherein the cosmetically-acceptable vehicle comprises a hydrophilic diluent.

39. The personal care composition of claim 1 wherein the cosmetically-acceptable vehicle comprises an emulsifier.

40. The personal care composition of claim 1 wherein the cosmetically-acceptable vehicle comprises an emollient.

41. The personal care composition of claim 1 wherein the cosmetically-acceptable vehicle comprises a silicone-based ingredient.

42. The personal care composition of claim 1 wherein the cosmetically-acceptable vehicle comprises a thickening agent.

43. The personal care composition of claim 1 wherein the cosmetically-acceptable vehicle comprises a humectant.

44. The personal care composition of claim 1 wherein the cosmetically-acceptable vehicle comprises a sunscreen ingredient.

45. The personal care composition of claim 1 wherein the personal care composition has an apparent viscosity of from about 5,000 to about 2,000,000 mPa.s.

46. The personal care composition of claim 1 wherein the personal care composition is aqueous.

47. The personal care composition of claim 1 wherein the personal care composition comprises an emulsion.

48. The personal care composition of claim 47 wherein the personal care composition comprises an oil-in-water emulsion.

49. The personal care composition of claim 47 wherein the personal care composition comprises a water-in-oil emulsion.

50. The personal care composition of claim 1 further comprising a long chain alcohol.

51. The personal care composition of claim 50 wherein the long chain alcohol has an average number of carbon atoms in the range of from about 8 to about 36.

52. A method of caring for skin or hair comprising applying to skin or hair the personal care composition of claim 1.

53. The personal care composition of claim 1 wherein said branched primary alcohol component comprises up to about 3 moles of alkylene oxide per mole of alcohol

54. The personal care composition of claim 53 wherein the branched primary alcohol component comprises from about 1 to about 3 moles of alkylene oxide per mole of alcohol.

55. The personal care composition of claim 53 wherein the alkylene oxide is selected from the group consisting of ethylene oxide, propylene oxide butylene oxide, and mixtures thereof.

56. The personal care composition of claim 53 wherein the alkylene oxide comprises ethylene oxide.

57. The personal care composition of claim 53 wherein the composition comprises about 5% or less by weight of surfactant.

58. The personal care composition of claim 53 wherein the average number of branches per molecule in the branched alcohol component is in the range from about 1.0 to about 3.0.

59. The personal care composition of claim 58 wherein the average number of branches per molecule in the branched primary alcohol component is in the range from about 1.5 to about 2.3.

60. The personal care composition of claim 53 wherein the branched primary alcohol component comprises less than about 0.5 atom % of quaternary carbon atoms.

61. The personal care composition of claim 53 wherein in the branched primary alcohol component less than about 5% of the alcohol molecules are linear alcohols.

62. The personal care composition of claim 53 wherein in the branched primary alcohol component at least about 40% of the number of branches in the alcohol are methyl branches.

63. The personal care composition of claim 53 wherein in the branched alcohol component from about 5% to about 30% of the number of branches in the alcohol are ethyl branches.

64. The personal care composition of claim 53 wherein the personal care composition comprises an emulsion.

65. The personal care composition of claim 64 wherein the personal care composition comprises an oil-in-water emulsion.

66. The personal care composition of claim 64 wherein the personal care composition comprises a water-in-oil emulsion.

67. A method of caring for skin or hair comprising applying to skin or hair the personal care composition of claim 53.

68. The personal care composition of claim 1 wherein said branched primary alcohol component comprises a product made by reacting alkylene oxide with branched primary alcohol in a ratio of up to about 3 moles of alkylene oxide per mole of alcohol.

69. The personal care composition of claim 68 wherein the branched primary alcohol component comprises a product made by reacting alkylene oxide with branched primary alcohol in a ratio of from about 1 to about 3 moles of alkylene oxide per mole of alcohol.

70. The personal care composition of claim 68 wherein the alkylene oxide is selected from the group consisting of ethylene oxide, propylene oxide butylene oxide, and mixtures thereof.

71. The personal care composition of claim 68 wherein the alkylene oxide comprises ethylene oxide.

72. The personal care composition of claim 68 wherein the composition comprises about 5% or less by weight of surfactant.

73. The personal care composition of claim 68 wherein the average number of branches per molecule in the branched alcohol component is in the range from about 1.0 to about 3.0.

74. The personal care composition of claim 73 wherein the average number of branches per molecule in the branched primary alcohol component is in the range from about 1.5 to about 2.3.

75. The personal care composition of claim 68 wherein the branched primary alcohol component comprises less than about 0.5 atom % of quaternary carbon atoms.

76. The personal care composition of claim 68 wherein in the branched primary alcohol component less than about 5% of the alcohol molecules are linear alcohols.

77. The personal care composition of claim 68 wherein in the branched primary alcohol component at least about 40% of the number of branches in the alcohol are methyl branches.

78. The personal care composition of claim 68 wherein in the branched alcohol component from about 5% to about 30% of the number of branches in the alcohol are ethyl branches.

79. The personal care composition of claim 68 wherein the personal care composition comprises an emulsion.

80. The personal care composition of claim 79 wherein the personal care composition comprises an oil-in-water emulsion.

81. The personal care composition of claim 79 wherein the personal care composition comprises a water-in-oil emulsion.

82. A method of caring for skin or hair comprising applying to skin or hair the personal care composition of claim 68.
